(19) United States
(12) Patent Application Publication
(10) Pub. No.: US 2007/0299456 A1
(43) Pub. Date: Dec. 27, 2007

(54) Light responsive medical retrieval devices

(76) Inventor: James A. Teague, Spencer, IN (US)

Correspondence Address: MAYER & WILLIAMS PC, 251 NORTH AVENUE WEST, 2ND FLOOR, WESTFIELD, NJ, 07090, US

(21) Appl. No.: 11/447,505

(22) Filed: Jun. 6, 2006

(51) Int. Cl. A61B 17/22

(52) U.S. Cl. 606/127

(57) ABSTRACT

A medical retrieval device comprising an elongate member that comprises a shape memory polymer. The elongate member undergoes a change in shape upon activation of the shape memory polymer with activating light.

### FIELD OF THE INVENTION

This invention relates to devices for the retrieval of concretions and other matter from the body of a subject.

### BACKGROUND OF THE INVENTION

Minimally invasive medical procedures generally involve causing limited trauma to the tissues of a subject. For example, minimally invasive surgical retrieval devices may be used to retrieve concretions, which can develop in various cavities within the body, for example, within the cavities of the kidneys, pancreas, and gallbladder. These devices may be used through an endoscope or a laparoscope, or they may be used without the aid of an endoscope or a laparoscope. Certain procedures utilize minimally invasive surgical retrieval devices, which have a sheath and a basket formed from wires. The basket is moveable in and out of the sheath and may be collapsed within the sheath to achieve a reduced radial profile. The basket may be opened when it extends beyond the distal end of the sheath or the sheath may be moved over the basket to collapse the basket.

Lithotripsy is a medical procedure that uses various forms of energy, such as acoustic shock waves, pneumatic pulsation, electrical hydraulic shock waves, or laser beams, to break up biological concretions such as urinary calculi (e.g., kidney stones) within the ureters. The force of the energy comminutes the calculi into smaller fragments that may be extracted from the body or allowed to pass through urination. In vivo fragmentation of urinary calculi can prove problematic in that stones and/or stone fragments within the ureter may become repositioned closer to and possibly migrate back into the kidney, thereby indicating further medical intervention to prevent the aggravation of the subject's condition.

### SUMMARY OF THE INVENTION

According to an aspect of the invention, medical retrieval devices are provided which comprise an elongate member that comprises a shape memory polymer. The elongate member undergoes a change in shape upon activation of the shape memory polymer with activating light.

An advantage of certain embodiments of the invention is that medical retrieval devices may be provided, which can radially expand in vivo without the aid of a sheath or other compressive article.

Another advantage of certain embodiments of the invention is that medical retrieval devices may be provided, which can radially contract or move in a fashion that facilitates withdrawal of the devices.

These and other aspects, embodiments, and advantages of the invention will be more fully understood by those of ordinary skill in the art upon review of the Detailed Description and Claims to follow.

### BRIEF DESCRIPTION OF THE DRAWINGS

FIGS. 1A and 1B are partial schematic perspective views of a retrieval assembly, in accordance with an embodiment of the invention.

FIG. 2 is a partial schematic perspective view of a retrieval assembly, in accordance with another embodiment of the invention.

FIG. 3A is a partial schematic perspective view of a retrieval/backstop assembly, in accordance with another embodiment of the invention.

FIG. 3B is a schematic perspective view of a central portion of the retrieval device of FIG. 3A, shown disposed over a guide wire, in accordance with an embodiment of the invention.

### DETAILED DESCRIPTION

According to an aspect of the invention, medical retrieval devices are provided which comprise an elongate member that comprises a shape memory polymer. The shape memory polymer is programmed such that it (and thus the elongate member which it forms, or of which it is a part) undergoes a change in shape upon activation with activating light (i.e., light such as visible light or ultraviolet light, which is of a frequency and intensity that is sufficient to activate the shape memory polymer). Such devices may be used to remove concretions or other materials from body cavities of subjects. Subjects include vertebrate animals, more typically mammals, and even more typically humans.

A near infinite variety of shape changes may be exhibited by the medical retrieval devices of the invention. For example, the elongate members may straighten upon activation with light, allowing the medical retrieval devices to be more readily moved within cavities (e.g., body cavities, device cavities, etc.) and/or allowing them to disengage from concretions, among other benefits. As another example, the elongate members may bend upon activation with light, for example, taking on a bowed, helical, or other configuration, allowing the medical retrieval devices to more readily capture concretions or prevent their migration.

In some embodiments of the invention, the elongate members are formed essentially entirely of shape memory polymer. In other embodiments, the elongate members are composite in character, being formed from at least one shape memory polymer and at least one additional material. In these embodiments, activation with light changes the shape of the shape memory polymer, which in turn changes the shape of the additional material with which it is associated. For example, the additional material may be an elongate metallic material such as nitinol, titanium or stainless steel, among many other possibilities. It may take, for instance, the form of a metallic wire (which may be, e.g., circular, oval, square, rectangular, etc., in cross-section), among other forms. In these embodiments, the shape memory polymer may extend along at least a portion of the length of the elongate metallic material, for example, extending along one side of the metallic material or extending completely around the circumference of the metallic material.

In some embodiments, the medical retrieval devices of the invention will contain one or more cavities. For example, the medical retrieval devices may contain one or more hollow elongate members (e.g., tubular elongate members), or the device may contain one or more cavities surrounded by one or more elongate members, among many other options. In such embodiments, the activating light may be directed on the inner surfaces of the devices (e.g., the cavity surfaces), on the outer surfaces of the devices, or both.

Where activating light is shown on inner surfaces of the devices, sources of activating light may be, for example, mounted within the retrieval devices or inserted into the retrieval devices. Where shown on outer surfaces of the devices, sources of activating light may, for example, be mounted on the outside surfaces of the retrieval devices or may be inserted separately from the retrieval device.

The retrieval devices may thus include one or more sources of activating light; or the activating light may be provided by separate devices which work in tandem with the retrieval devices.

The retrieval devices may work in tandem with scopes, such as endoscopes or laparoscopes, or they may work independently of such devices.

Materials, including shape memory polymers, are said to show a shape memory effect if they can be deformed and fixed into a temporary shape, and subsequently approach or completely return to their original (or “permanent”) shape upon exposure to an activating stimulus.

As used herein, a shape memory polymer is a material which has shape memory and which contains polymer molecules, for example, containing 50 wt % to 75 wt % to 90 wt % to 95 wt % to 99 wt % or more polymer molecules. As used herein, “polymer molecules” are molecules that contain multiple copies of one or more types of constitutional units, commonly referred to as monomers, and typically contain from 5 to 10 to 25 to 50 to 100 to 500 to 1000 or more constitutional units. Polymer molecules may be, for example, homopolymer molecules, which contain multiple copies of a single constitutional unit, or copolymer molecules, which contain multiple copies of at least two dissimilar constitutional units, which units may be present in any of a variety of distributions including random, statistical, gradient, periodic (e.g., alternating), and block distributions, among others. Polymer molecules may have a variety of architectures, including cyclic, linear, branched and networked architectures, among others. Polymer molecules may be crosslinked, as described below.

With shape memory polymers, the process of programming and recovery of a shape is generally as follows: (a) first, the polymer is conventionally processed to receive its original, or permanent, shape, (b) the polymer is then deformed and the intended temporary shape is fixed in a process called programming. The permanent shape is now stored while the polymer displays the temporary shape. Subsequent activation of the shape memory polymer using a suitable recovery stimulus causes the polymer to approach (up to and including complete recovery of) the stored, permanent shape.

Shape memory polymers that utilize light for as an activation stimulus are known. For example, A. Lendlein et al., Nature, Vol. 434, 14 Apr. 2005, 879-882, report polymers containing cinnamic groups which can be deformed and fixed into a temporary shape by exposure to ultraviolet light illumination of a first wavelength (i.e., λ>260 nm). The polymers can subsequently recover their original shape at upon exposure to ultraviolet light of a different wavelength (i.e., λ<260 nm). Without wishing to be bound by theory, according to Lendlein et al., the photoresponsive shape memory polymers described therein contain the following: (a) so-called “molecular switches” that fix the temporary shape by forming crosslinks, specifically, photoresponsive cinnamic acid type molecules such as cinnamic acid (CA) and cinnamylidene acetic acid (CAA), which are able to undergo efficient photoreversible reactions when exposed to alternating wavelengths and (b) so-called ‘netpoints’ that determine the permanent shape of the polymer, in particular, a covalently crosslinked, amorphous, permanent polymer network. When the photoresponsive polymer is stretched, the coiled segments of the amorphous polymer chains between netpoints are elongated. Upon exposure to UV light of >260 nm, the coiled segments are partially fixed in the elongated state due to the formation of new photoresponsive crosslinks, resulting in a deformed new shape. When the photoresponsive crosslinks are reversibly cleaved by irradiation with UV light of <260 nm, the fixed elongated film shape shifts toward, or achieves, its original permanent shape. Two photoresponsive shape-memory polymers are created in Lendlein et al., one in which CA molecules are grafted onto a permanent polymer network (i.e., n-butylacrylate (BA), hydroxyethyl methacrylate (HEMA) and ethyleneglycol-1-acrylate-2-CA (HEA-CA), are copolymerized and crosslinked with poly(propylene glycol)-dimethacrylate) and one in which CAA are provided in an interpenetrating network polymer (i.e., a permanent network is formed from poly(n-butyl acrylate) using poly(propylene glycol)-dimethacrylate as a crosslinker, and loaded with star-poly(ethylene glycol) containing CAA terminal groups). See also U.S. patent No. 2003/0055198 to R. S. Langer and A. Lendlein.

Regardless of the type of shape memory polymer employed or its precise mechanism of operation, light actuated shape memory polymers may be incorporated into a variety of medical retrieval devices in accordance with the invention.

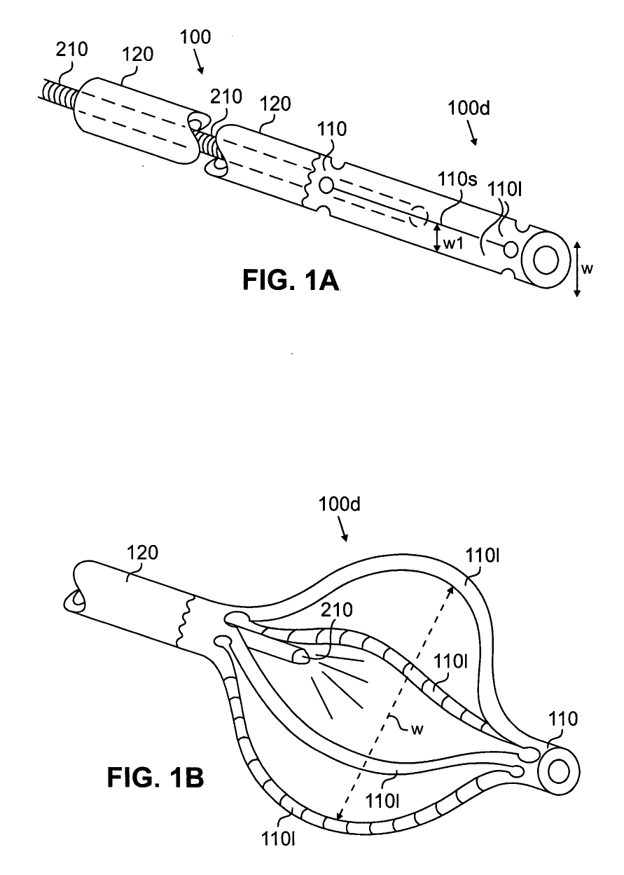

For example, FIG. 1A is a schematic perspective view of a light-actuated retrieval device 100 in accordance with an embodiment of the present invention. The retrieval device 100 can be, for example, used in ureteroscopy procedures, among others. At the distal end 100d of the device 100 there is provided a shape memory portion 110 having a plurality of elongate members, specifically legs 110l, which extend axially along the length of the device 100. Legs 110l may be formed, for example, by cutting (e.g., die cutting, laser cutting, etc.) a series slots 110s along a length of shape memory polymer tubing. Although the device 100 shown contains four slots and four legs, other numbers of slots and legs (e.g., 3, 5, 6, etc.) can clearly be formed to achieve an analogous effect. A hole is formed at the end of each slot in the embodiment shown for purposes of relieving stress at both ends of the slots preventing slot migration or “ripping”, although this is not essential.

The shape memory portion 110 is disposed at the end of an elongate delivery member, such as a delivery tube 120, which is used to advance the shape memory portion 1 10 to a desired position within the body of a subject. The length of the elongate delivery member will vary from application to application. For example, where used in ureteroscopy procedures, the elongate delivery member may have an overall length that is sufficient to advance the shape memory portion 110 to the kidney. The material forming the elongate delivery member will have a strength and flexibility suitable for the application at hand. For example, where used in ureteroscopy procedures, the elongate delivery member may be formed from a polymeric material such as ethylene/vinyl acetate copolymer (EVA), polyurethane (PU), and high to low density polyethylene (HDPE to LDPE), among many other possibilities.

Various devices can be advanced through the lumens of the delivery tube 120 and the shape memory portion 110. For example, an imaging assembly (e.g., one that includes a source of radiation and a sensor for sensing reflected radiation) may be inserted through the lumens to visualize a concretion that is to be removed. For example, a radiation source, such as a light source, may be disposed at a distal end of an elongated delivery member, such as a rod or tube, or illumination from a light source may be coupled to a light guide (e.g., an optical fiber or fiber bundle, with an optional diffuser) and carried to the distal end of the device 100 via the light guide. A sensor, for example, a camera such as a CCD camera chip or CMOS camera chip, may be disposed at a distal end of a separate elongated delivery member, such as a rod or tube. Or both the radiation source and the sensor may carried by a single elongated delivery member, among other possibilities.

As another example, an energy source such as a laser, fiber, or pneumatic probe may be advanced through the lumen of the delivery tube 120 in order to break up a concretion during the course of a lithotripsy procedure.

As yet another example, and as illustrated in FIGS. 1A and 1B, an activation device 210 may be inserted through the lumen of the delivery tube 120 so as to direct activating light onto the shape memory polymer 110 and allow it to revert toward its permanent shape. For example, the activation device 210 may contain a light guide, which carries activating light along its length from a light source (e.g., a lamp, laser, diode, laser diode, etc.) that is optically coupled to its proximal end. The light guide may be, for example, an optical fiber (shown in FIGS. 1A and 1B), such as a quartz-based optical fiber among others, or an optical fiber bundle, either of which may have an optional diffuser disposed at its the distal end. Alternatively the activation device may comprise, for example, an elongate delivery member (e.g., rod, tube, etc.) that carries a light source at its distal end.

In other alternatives, the activation device may be integrated into the retrieval device 100. For example one or more light sources may be provided within the device 100, for example, by mounting the light source within the lumen of the device such that it directs activating light onto the inside surface of the elongated members.

In yet other alternatives, at least one light source may be provided outside the device, for example, by integrating the at least one light source into an endoscope or laparoscope that is used in conjunction with the retrieval device, or by mounting the at least one light source on an outer surface of the retrieval device, such that it shines on the outside surface of the elongated members. Such a light source (or sources) may, for example, supplement an internally integrated or internally inserted light source (or sources) such as those described in the prior paragraph, or it (they) may be used as an alternative to the same.

Turning back to FIGS. 1A and 1B, as seen in FIG. 1B, upon illumination of the interior surface of the shape memory polymer 110 with activating light from the activation device 210, the shape memory polymer moves toward its original shape in which the elongate members (i.e., legs 110l) bow radially outward from the device. When this procedure is performed in vivo, a “basket” is effectively formed within the body cavity (e.g., a body lumen such as a ureter) into which it has been inserted. By deploying the basket distal to a concretion such as a kidney stone, the basket may capture the concretion as it is withdrawn from the body, pulling the concretion along with it.

In general, larger body cavities dictate the need for larger device widths w, and vice versa. Moreover, an increase in length of the elongate members (i.e., legs 110l) results in an increase in the width w that can be achieved for the device, and vice versa. The width of the legs 110l themselves w1 is dictated, for example, by the number and placement of the slots.

Although the basket of FIG. 1B is formed using elongate members 110l whose proximal and distal ends are affixed to the main tubular body of the device, in other embodiments, one end of each the elongate member (e.g., the distal end) may be free.

Typical retrieval baskets are metallic and have preformed shapes, which have diameters that are reduced by collapsing the baskets within sheaths. Once the sheathed baskets are positioned in a body lumen beyond a concretion, the baskets are opened (or closed) with the aid of the sheaths (e.g., by distally moving the baskets relative to the sheaths). One drawback to such devices is that pre-formed metallic geometries require heavy walled sheaths in order to collapse the baskets into the same. This increases the minimum overall diameter of the device.

Expandable devices in accordance with the present invention, on the other hand, do not require a delivery sheath in all embodiments. For example, devices in accordance with the invention, may be formed such that the original (or “permanent”) shape of the elongate member or members is a radially expanded shape (see, e.g., the shape of FIG. 1B, among many possibilities). The devices may then be placed in a radially compressed state. For example, referring to a device having a permanent shape like that of FIG. 1B, the ends of the shape memory portion 110 may be pulled in opposite directions to radially collapse the legs 110l, or the legs 110l may be collapsed using a temporary sheath, among other techniques, thereby forming a radially collapsed structure (e.g., a structure like that of FIG. 1A whose overall width w is reduced to the overall width of the expanded structure of FIG. 1B). While in this collapsed state, the shape memory polymer may be suitably processed, for instance, exposed to light of a suitable wavelength and intensity on its internal surface, its external surface (e.g., using a transparent sheath, where a temporary sheath is employed), or both, so as to temporarily affix the shape memory portion 110 in a collapsed shape. Subsequently, the temporary structure may be exposed to activating light on its internal surface, its external surface, or both, to radially expand it back toward its permanent shape. In general, the same surfaces that are exposed to light to fix the temporary shape are also exposed to light to return to the permanent shape. 036 Other embodiments of the invention, in contrast, do utilize delivery sheaths. For example, devices in accordance with the invention may be formed in which the permanent shape of the shape memory portion 110 is a radially contracted shape (see, e.g., FIG. 1A, among many possibilities) and in which the temporary shape is a radially expanded shape (see, e.g., FIG. 1B). This latter structure may be compressed into a sheath and inserted into a subject as described below.

Such a device may be formed, for example, by longitudinally stretching a shape memory polymer tube 110 like that of FIG. 1A and shining light on the outside surface of the tube (i.e., the slotted region of the tube), which is of a wavelength and intensity suitable to form a temporary shape. As described in Lendlein et al. above, although the deformation is well-fixed for those portions of the shape memory polymer that have been exposed to light (the material at the outer surface of the legs), the material that has not been exposed to light (the material at the inner surface of the legs) keeps its elasticity. Consequently, the material at the inner surface contracts much more than the material at the outer surface once the external stress is removed, which can form an arched or bowed member. Subsequently activating light may be shined on the outer surface of the device (e.g., on its outer surface) to recover the permanent, radially contracted state.

As another example, an expandable device (e.g., a balloon, etc.) may be placed within a multi-leg device like that of FIG. 1A (in particular, within the slotted region of the shape memory polymer tube 110) and expanded (e.g., by inflation, etc.) to outwardly bow the legs into a radially expanded state like that of FIG. 1B. Light is then shown on the legs (e.g., the inside surfaces, the outside surfaces, the sides, and combinations thereof) to fix the member in an expanded state.

Regardless of how the device 100 is provided with a temporary, radially expanded state, in such embodiments, the distal end of the device 100 may be compressed and inserted into a delivery sheath. Forming the legs entirely of shape memory polymer decreases the columnar strength requirement of the sheath, and therefore may down-size the device, allowing the device to be guided into tight body cavities. After advancing the sheathed device into a subject, an expanded configuration may be formed in vivo (e.g., distal to a concretion such as a kidney stone) by retracting the sheath, whereupon the device may capture the concretion as it is withdrawn from the body. In some instances, however, difficulties may be encountered in removing the concretion, which also prevents the device 100 from being retracted and/or removed altogether from the body cavity. In the present embodiment of the invention, however, the basket may be moved distal to the stone, followed by exposure to activation light such that the shape memory portion 110 returns toward its permanent, radially contracted state for easier withdrawal.

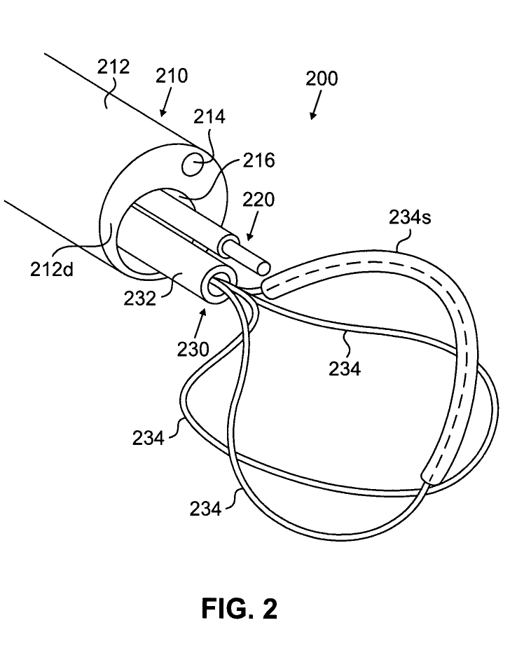

Other embodiments of the invention will now be described in conjunction with the retrieval assembly 200 illustrated in FIG. 2. The retrieval assembly 200 includes a scope portion 210, an activation device 220 and a retrieval device 230. The scope portion 210 may be any suitable scope portion such as those known in the art, including endoscopes, laparoscopes, and so forth. The scope portion 210 may comprise, for example, an extrusion 212, having a major lumen 216 through which, for example, the activation device 220 and the retrieval device 230 may be inserted, and a minor lumen 214 through which, for example, an optical fiber or hard wired CCD chip may be inserted. The extrusion may be formed, for example, using polyurethane or polyethylene, among numerous other thermoplastic polymers. Of course, the retrieval device 230 and the activation device 220 may be operated in conjunction with other differing endoscope designs or even without an endoscope in certain embodiments of the invention (e.g., by integrating the activation device 220 into the retrieval device 230, or by using separate devices 220, 230).

The activation device 220 may be any suitable activation device, including those described hereinabove. The activation device 220 in the embodiment shown is an optical fiber having a light source (e.g., a UV light source) coupled to its proximal end, although a light source may be placed at the distal end of the 212d of the extrusion 212, or at the distal end of an elongate delivery member (e.g., a rod, tube, etc.), without the need for a waveguide, among other possibilities. As above, a diffuser may be optionally provided to scatter light emerging from the fiber(s). Moreover, rather than one fiber, a bundle of fibers may be used, among other options.

The retrieval device 230, partially resembles “zero-tip” retrieval devices such as those described in U.S. Pat. Nos. 6,302,895, 6,159,220, 6,527,781 and 6,224,612 and includes the following components: (a) an elongate delivery member 232 (e.g., a rod, tube, etc.) which may be formed from polytetrafluoroethylene (PTFE) or another suitable polymer or may be a composite sheath made from an inner layer of PTFE, wire braiding, and a stiff outer layer of polyimide, and (b) multiple legs 243 which may be secured to the distal end of elongate delivery member 232 or advanced through a lumen of elongate delivery member 232 (e.g., where the elongate delivery member 232 functions as a sheath). The legs 243 may be formed from a material such as nitinol, which is capable of being compressed (e.g., into an endoscope lumen, or into a sheath) and which springs back to its original shape (e.g., after emerging from the lumen or sheath). Although 4 legs are illustrated, other numbers of legs may be used (e.g., 3, 5, 6, etc.).

Unlike known “zero-tip” retrieval devices, however, at least one of the legs 234s (one shown) comprises a shape memory polymer. For example, the at least one leg 234s may be formed entirely from the shape memory polymer, or it may be a composite structure comprising the shape memory polymer and another material. For example, as illustrated in FIG. 2, the leg 234s is similar to the other legs 234, except that it is covered by a shape memory polymer, thereby allowing the user to reshape the leg 234s with light from the activation device 220. The shape memory polymer covering may correspond, for instance, to a straight length of shape memory polymer tubing (the permanent shape). This polymer tubing may then be bent and fixed in a temporary bent configuration by shining light of a suitable wavelength and intensity on the polymer tubing as discussed above, with the result being a configuration like that shown in FIG. 2.

The retrieval assembly 200 of the device of FIG. 2 may be operated as follows. First, the endoscope portion 210 may be inserted until it lies just proximal to a concretion. Then, the retrieval device 230 is advanced (either with or without a delivery sheath, as needed) and deployed as illustrated in FIG. 2. The bent shape of the leg 234s is due to the memorized or temporary shape of the shape memory polymer. In a subsequent step, activating light is directed from the activation device 220 onto the shape memory polymer, returning it to its permanent shape. In the particular embodiment shown, this results in a straightening of the leg 234s, which acts to tilt the retrieval device off-axis with respect to the endoscope.

Such a device may be useful, for example, where a concretion is difficult to reach because it is off-axis from the device. This occurs, for example, when a calculus is formed in the calyx of the kidney. The physician often finds it difficult to place the retrieval device into the small irregularly shaped cavities that are off plane to the device.

A device of this type may also be useful, for example, where a concretion becomes trapped in the basket, for example, giving the user the ability shift the retrieval device remotely in an attempt to release the stone from the basket.

In certain embodiments, including various embodiments already described elsewhere herein, devices in accordance with the present invention may be used to immobilize concretions while they are being reduced in size, for example, during lithotripsy. As noted above, lithotripsy is a medical procedure that uses energy in various forms such as acoustic shock waves, pneumatic pulsation, electrical hydraulic shock waves, or laser beams to break up biological concretions such as urinary calculi (e.g., kidney stones). In some of these embodiments, for example, the distal portion of the retrieval device may form a backstop and may prevent the advancement of the stone or remnants thereof toward the kidney during lithotripsy. The backstop may also enable a user to increase the amount of energy supplied to the stone during the procedure.

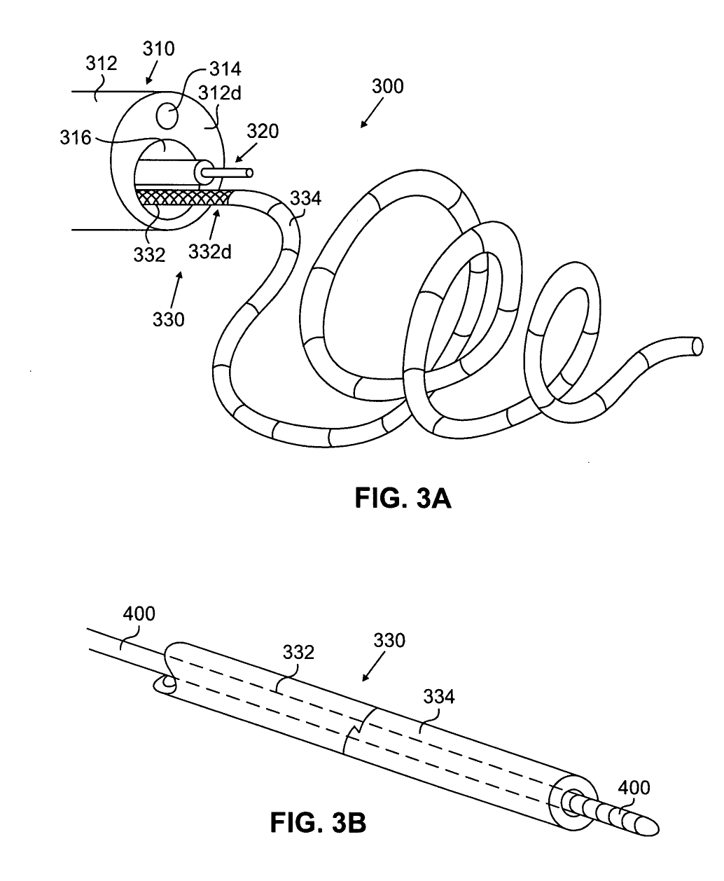

One specific example of such a device will now be described in conjunction with FIG. 3A, in which a retrieval assembly 300 is illustrated. The retrieval assembly 300 includes an endoscope portion 310, an actuation device 320, and a retrieval device 330. The actuation device 320 and the endoscope portion 310 (which includes an extrusion 312, displaying at its distal end 312d a major lumen 316 and a minor lumen 314) are substantially like those in FIG. 2, as illustrated, and numerous variations concerning the actuation device 320 and the endoscope portion 310 described in conjunction with those figures are applicable here as well.

The retrieval device 330 includes a shape memory portion 334 that is attached to the distal end 332d of an elongate delivery member 332 (e.g., a rod, a tube, etc.) which may be formed, for example, from extruded copolymer like polyethylene (e.g., HDPE) or another suitable polymer, among other materials. The shape memory portion 334 may be formed entirely from a shape memory polymer, or it may be a composite of a shape memory polymer and another material.

The retrieval device 330 is illustrated in FIG. 3A in its deployed orientation. The retrieval device 330 may be formed, for example, such that the original (or “permanent”) shape of the shape memory portion 334 is a fully deployed shape (e.g., in the form of the conical helix of FIG. 3A, among many possibilities).

Once formed, each end of this “permanent” structure may be pulled in opposite directions to form a more linear structure. This structure is then exposed to light which is of a wavelength and intensity suitable to temporarily fix the shape memory portion 334 in the more linear form.

The retrieval assembly 300 of FIG. 3A may be operated as follows. First, the endoscope portion 310 may be inserted into the subject until is lies proximal to a concretion. Then, the retrieval device 330, which comprises the shape memory portion 334 in its more linear shape, may be advanced through the endoscope lumen 316 until the shape memory portion 334 of the retrieval device 330 lies distal to the stone.

In some embodiments, the retrieval assembly 300 may be inserted over a guide wire. A central portion of such a retrieval device 330 is illustrated in FIG. 3B, in which a portion of the elongate delivery member 332 and a portion of the shape memory portion 334 (in a more linear shape) are shown. The retrieval device 330 of FIG. 3B is of a tubular construction and is shown deployed over a guide wire 400, which may be formed, for example, from wound stainless steel over a stainless steel or nitinol core wire, among various other materials and designs.

Subsequent to advancement, activating light is directed from the activation device 320 onto the shape memory portion 334, causing the shape memory portion 334 to revert to its permanent shape (e.g., a radially expanded shape, such as the conic helix form of FIG. 3A, among many possibilities). Once the retrieval device 330 is deployed, a lithotripsy procedure may be performed if desired (e.g., by inserting a lithotripsy device through the major lumen, for example, after withdrawing the activation device 320), after which the stone (or its remnants) may be removed from the subject by withdrawal of the retrieval device 330.

Because the shape memory portion 334 forms a “basket” (e.g., one having a conical helix shape, like that of FIG. 3A, among other possibilities), the device 330 of FIG. 3A may also be used as a retrieval device, even in the absence of a lithotripsy procedure. Where the permanent shape is such that the basket is tilted off-axis, such a device may be useful, for example, where the concretion is in a difficult to reach cavity, such as the calyx of the kidney.

In other embodiments, the permanent form of the shape memory portion 334 is substantially linear, and the temporary form of the shape memory portion 334 is a radially expanded form such as a conical helix. Such a device 330 may be formed, for example, by first deforming a substantially linear shape memory portion 334 into a radial expanded shape. This structure is then exposed to light which is of a frequency and intensity suitable to fix the shape memory portion 334 in the radially expanded shape.

In order to advance the device 330, the shape memory portion 334 may be compressed into a sheath. Once the device 330 is properly positioned, the sheath may be retracted to allow the shape memory portion 334 to regain its radially expanded shape. Once deployed, the device 330 may be used to capture and remove concretions. As previously noted, in some instances, difficulties may be encountered in removing concretions, for example, because they are too large. In the present embodiment of the invention, however, the basket may be exposed to activating light, for example, by advancing an activation device through an endoscope lumen (where used), by advancing an activation device through an internal lumen of the device 330 (if present), by advancing an activation device in a manner that is independent of another device, and so forth. After exposure to activating light, the shape memory portion 334 reverts toward its permanent, more linear state for easier withdrawal.

Although various embodiments are specifically illustrated and described herein, it will be appreciated that modifications and variations of the present invention are covered by the above teachings and are within the purview of the appended claims without departing from the spirit and intended scope of the invention.

1. A medical retrieval device comprising an elongate member that comprises a shape memory polymer, said elongate member undergoing a change in shape upon activation of said shape memory polymer with activating light.

2. The medical retrieval device of claim 1, wherein said elongate member consists essentially of a shape memory polymer.

3. The medical retrieval device of claim 1, wherein said elongate member comprises a shape memory polymer and an additional material.

4. The medical retrieval device of claim 3, wherein said additional material is a metallic material.

5. The medical retrieval device of claim 3, wherein said additional material is nitinol.

6. The medical retrieval device of claim 3, wherein said additional material is a metallic wire.

7. The medical retrieval device of claim 6, wherein said a shape memory polymer extends along at least a portion of the length of said metallic wire.

8. The medical retrieval device of claim 7, wherein said shape memory polymer wraps around said metallic wire.

9. The medical retrieval device of claim 1, wherein said shape member polymer undergoes a change in shape upon activation with ultraviolet light.

10. The medical retrieval device of claim 1, wherein said medical device comprises a plurality of said elongate members.

11. The medical retrieval device of claim 1, wherein said activating light is directed on an outer surface of said elongate member to activate said shape memory polymer.

12. The medical retrieval device of claim 11, where said medical retrieval device comprises a source of activating light for activation of said shape memory polymer.

13. The medical retrieval device of claim 1, wherein said medical retrieval device comprises an inner cavity.

14. The medical retrieval device of claim 13, wherein said cavity corresponds to a hollow elongate member.

15. The medical retrieval device of claim 13, wherein said medical device comprises a plurality of said elongate members

16. The medical retrieval device of claim 13, wherein said cavity is surrounded by a plurality of said elongate members.

17. The medical retrieval device of claim 13, wherein said activating light is directed on an inner surface of said cavity to activate said shape memory polymer.

18. The medical retrieval device of claim 17, where said device comprises a source of activating light that is configured to direct said activating light on said inner surface of said cavity.

19. The medical retrieval device of claim 13, wherein said activating light is directed on an outer surface of said device to activate said shape memory polymer.

20. The medical retrieval device of claim 19, where said device comprises a source of activating light that is configured to direct said activating light on said outer surface of said device.

21. The medical retrieval device of claim 13, wherein said activating light is directed on an inner surface of said cavity and on an outer surface of said device to activate said shape memory polymer.

22. The medical retrieval device of claim 1, wherein said elongate member straightens upon activation with said activating light.

23. The medical retrieval device of claim 1, wherein said elongate member bends upon activation with said activating light.

24. The medical retrieval device of claim 23, wherein said elongate member becomes bowed upon activation with said activating light.

25. The medical retrieval device of claim 23, wherein said elongate member forms a conic helix upon activation with said activating light

26. A kit comprising the medical device of claim 1 and a delivery sheath that compresses said elongated member and reduces the diameter of said retrieval device.

27. A kit comprising the medical device of claim 1 and a device that supplies said activating light.

28. The kit of claim 27, wherein said device that supplies said activating light comprises an activating light source coupled to a fiber optic or a bundle of fiber optics.
